(19) United States
(12) Patent Application Publication
(10) Pub. No.: US 2013/0273138 A1
(43) Pub. Date: Oct. 17, 2013

(54) TRANSDERMAL FORMULATIONS OF GERANYLGERANYLACETONE

(71) Applicant: COYOTE PHARMACEUTICALS, INC., Menlo Park, CA (US)

(72) Inventor: HIROAKI SERIZAWA, Menlo Park, CA (US)

(21) Appl. No.: 13/779,565

(22) Filed: Feb. 27, 2013

Related U.S. Application Data
(60) Provisional application No. 61/605,155, filed on Feb. 29, 2012.

(51) Int. Cl. A61K 9/70; A61K 9/00; A61K 31/121

(52) U.S. Cl. CPC A61K 9/7023; A61K 31/121; A61K 9/0014
USPC 424/443; 514/675

(57) ABSTRACT

Provided herein is a pharmaceutical formulation comprising at least one geranylgeranyl acetone in the form of a transdermal patch. Also provided herein are methods of treating neural diseases or disorders by administering such pharmaceutical formulations.

### CROSS REFERENCE TO RELATED APPLICATION

This application claims priority under 35 U.S.C. section 119(e) of U.S. provisional application no. 61/605,155 filed on Feb. 29, 2012, which is incorporated in its entirety herein by reference

### FILED OF THE INVENTION

This invention relates to a pharmaceutical formulation comprising at least one geranylgeranylacetone in the form of a transdermal patch, and also to methods of treating neural diseases or disorders by administering such pharmaceutical formulations.

### STATE OF THE ART

Geranylgeranylacetone (GGA) has the formula:

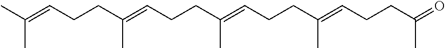 [SMILES: CC(=O)CC/C=C(\C)CC/C=C(\C)CC/C=C(\C)CCC=C(C)C]

and is reported to have neuroprotective and related effects. See, for example, PCT Pat. App. No. WO 2012/031028 which is incorporated herein by reference in its entirety. Neural diseases such as amyotropic lateral sclerosis (ALS), multiple sclerosis (MS), Alzheimer's disease (AD), and Parkinson's disease (PD) afflict the patients for a prolonged period of time. In many cases the patient is unable to reliably self administer medicaments for treating the disease. Therefore patient compliance is problematic at best. Furthermore, administration of the necessary medication to such patients requires the use of caregivers and increases the treatment costs. It would benefit the patients and the healthcare system if a suitable therapy could be administered to such patients for a prolonged period of time as necessary. Such would reduce the caregiver's burden and the costs associated therewith. However, not all drugs are suitable for sustained release as they are either not compatible for transdermal penetration or formulation. Specifically, it is necessary to formulate a transdermal composition such that the active drug has a transportability into and through the skin from its corresponding transdermal formulation. It has been found that GGA, and transdermal formulations of it, are capable of such prolonged delivery via the skin.

### SUMMARY OF THE INVENTION

It has been found that (5E, 9E, 13E) geranylgeranyl acetone is amenable to sustained and controlled release by transdermal technology and that such provides for long term therapy for patients benefiting from treatment with (5E, 9E, 13E) geranylgeranyl acetone. Accordingly, provided herein in some embodiments is a medical patch for the rate-controlled transdermal administration of (5E, 9E, 13E) geranylgeranyl acetone through intact skin for an extended period of time which comprises: (a) a drug reservoir comprising (5E, 9E, 13E) geranylgeranyl acetone and a permeation enhancer in amounts sufficient to deliver (5E, 9E, 13E) geranylgeranyl acetone at a therapeutically effective rate for said extended period of time; and (b) a drug-impermeable backing laminate. In some embodiments, (5E, 9E, 13E) geranylgeranyl acetone is present in a ratio of greater than 80:20 of (5E, 9E, 13E) to (5Z, 9E, 13E) geranylgeranyl acetone isomers. In other embodiments, (5E, 9E, 13E) geranylgeranyl acetone is present in a ratio of greater than 90:10 of (5E, 9E, 13E) to (5Z, 9E, 13E) geranylgeranyl acetone isomers. In specific embodiments, the permeation enhancer is ethanol, glycerol monocaprylate, glycerol monolaurate, glyceryl monooleate, hydroxypropyl β-cyclodextrin, lauric acid, myristic acid, oleic acid, oleyl alcohol, palmitic acid, polyethylene glycol, propylene glycol, sodium lauryl sulfate(SLS), steric acid, tween 80 or combination thereof

Some embodiments provided herein describe an apparatus for treatment of a neural disease, disorder or condition, comprising an adhesive patch for being adhered to the skin of a subject suffering from the neural disease, disorder or condition, a therapeutically effective amount of (5E, 9E, 13E) geranylgeranyl acetone wherein the compound is dispersed on said patch; and at least one permeation enhancer for introducing said compound from the patch into the body of the subject. In some embodiments, (5E, 9E, 13E) geranylgeranyl acetone is present in a ratio of greater than 80:20 of (5E, 9E, 13E) to (5Z, 9E, 13E) geranylgeranyl acetone isomers. In other embodiments, (5E, 9E, 13E) geranylgeranyl acetone is present in a ratio of greater than 90:10 of (5E, 9E, 13E) to (5Z, 9E, 13E) geranylgeranyl acetone isomers. In certain specific embodiments, the permeation enhancer is ethanol, glycerol monocaprylate, glycerol monolaurate, glyceryl monooleate, hydroxypropyl13-cyclodextrin, lauric acid, myristic acid, oleic acid, oleyl alcohol, palmitic acid, polyethylene glycol, propylene glycol, sodium lauryl sulfate (SLS), steric acid, tween 80 or combination thereof In some embodiments, the neural disease or condition is Alzheimer's disease, amyotrophic lateral sclerosis disease, Parkinson's disease, or multiple sclerosis.

Also provided herein in some embodiments is a method for treating a neural disease, disorder or condition, the method comprising administering to a subject suffering from said neural disease, disorder, or condition, a therapeutically effective amount of (5E, 9E, 13E) geranylgeranyl acetone, wherein (5E, 9E, 13E) geranylgeranyl acetone is administered to said subject by transdermal application.

Some embodiments provided herein describe a method for inducing expression of a heat shock protein in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application.

Other embodiments provided herein describe a method for inhibiting neural death in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application

In some embodiments, the (5E, 9E, 13E) geranylgeranyl acetone is essentially free of or is free of (5Z, 9E, 13E) geranylgeranyl acetone.

### BRIEF DESCRIPTION OF THE FIGURES

FIGS. 1A and 1B show two HPLC chromatograms (one standard of 0.05 mg/mL and one sample taken at 24 hour time point). Nearly all the GGA in the membrane was released according to this specific dissolution setting.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Certain Definitions

Unless otherwise noted, terminology used herein should be given its normal meaning as understood by one of skill in the art.

As used herein, the term “comprising” or “comprises” is intended to mean that the compositions and methods include the recited elements, but not excluding others. “Consisting essentially of when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed invention. “Consisting of” shall mean excluding more than trace elements of other ingredients and substantial method steps.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “C₁-C₆-alkyl” as used herein refer to saturated, straight- or branched-chain hydrocarbon radicals derived from a hydrocarbon moiety containing between one and three, one and six, and one and twelve carbon atoms, respectively, by removal of a single hydrogen atom. Examples of C₁-C₆-alkyl radicals include, but not limited to, methyl, ethyl, propyl, isopropyl, n-butyl, tert-butyl, neopentyl and n-hexyl.

The alkyl group may optionally be substituted by one or more of fluorine, chlorine, bromine, iodine, carboxyl, C₁₋₄ alkoxycarbonyl, C₁₋₄ alkylaminocarbonyl, di-(C₁₋₄ alkyl)-aminocarbonyl, hydroxyl, C₁₋₄ alkoxy, formyloxy, C₁₋₄ alkylcarbonyloxy, C₁₋₄ alkylthio, C₃₋₆ cycloalkyl or phenyl.

The term “aryl” as used herein, alone or in combination, refers to an optionally substituted aromatic hydrocarbon radical of six to about twenty ring carbon atoms, and includes fused and non-fused aryl rings. A fused aryl ring radical contains from two to four fused rings where the ring of attachment is an aryl ring, and the other individual rings may be alicyclic, heterocyclic, aromatic, heteroaromatic or any combination thereof Further, the term aryl includes fused and non-fused rings containing from six to about twelve ring carbon atoms, as well as those containing from six to about ten ring carbon atoms. A non-limiting example of a single ring aryl group includes phenyl; a fused ring aryl group includes naphthyl, phenanthrenyl, anthracenyl, azulenyl; and a non-fused bi-aryl group includes biphenyl.

The term “neuroprotective” refers to reduced toxicity of neurons as measured in vitro in assays where neurons susceptible to degradation are protected against degradation as compared to control. Neuroprotective effects may also be evaluated in vivo by counting neurons in histology sections.

The term “neuron” or “neurons” refers to all electrically excitable cells that make up the central and peripheral nervous system. The neurons may be cells within the body of an animal or cells cultured outside the body of an animal. The term “neuron” or “neurons” also refers to established or primary tissue culture cell lines that are derived from neural cells from a mammal or tissue culture cell lines that are made to differentiate into neurons. “Neuron” or “neurons” also refers to any of the above types of cells that have also been modified to express a particular protein either extrachromosomally or intrachromosomally. “Neuron” or “neurons” also refers to transformed neurons such as neuroblastoma cells and support cells within the brain such as glia.

The term “protein aggregates” refers to a collection of proteins that may be partially or entirely mis-folded. The protein aggregates may be soluble or insoluble and may be inside the cell or outside the cell in the space between cells. Protein aggregates inside the cell can be intranuclear in which they are inside the nucleus or cytoplasm in which they are in the space outside of the nucleus but still within the cell membrane. The protein aggregates described in this invention are granular protein aggregates.

As used herein, the term “protein aggregate inhibiting amount” refers to an amount of compound that inhibits the formation of protein aggregates at least partially or entirely. Unless specified, the inhibition could be directed to protein aggregates inside the cell or outside the cell.

As used herein, the term “intranuclear” or “intranuclearly” refers to the space inside the nuclear compartment of an animal cell.

The term “cytoplasm” refers to the space outside of the nucleus but within the outer cell wall of an animal cell.

As used herein, the term “pathogenic protein aggregate” refers to protein aggregates that are associated with disease conditions. These disease conditions include but are not limited to the death of a cell or the partial or complete loss of the neuronal signaling among two or more cells. Pathogenic protein aggregates can be located inside of a cell, for example, pathogenic intracellular protein aggregates or outside of a cell, for example, pathogenic extracellular protein aggregates.

As used herein, the term “SBMA” refers to the disease spinal and bulbar muscular atrophy. Spinal and bulbar muscular atrophy is a disease caused by pathogenic androgen receptor protein accumulation intranuclearly.

As used herein, the term “ALS” refers to amyotrophic lateral sclerosis disease.

As used herein, the term “AD” refers to Alzheimer's disease.

The term “neurotransmitter” refers to chemicals which transmit signals from a neuron to a target cell. Examples of neurotransmitters include but are not limited to amino acids such as glutamate, aspartate, serine, y-aminobutyric acid, and glycine; monoamines such as dopamine, norepinephrine, epinephrine, histamine, serotonin, and melatonin; and other molecules such as acetycholine, adenosine, anadamide, and nitric oxide.

The term “synapse” refers to junctions between neurons. These junctions allow for the passage of chemical signals from one cell to another.

The term “G protein” refers to a family of proteins involved in transmitting chemical signals outside the cell and causing changes inside of the cell. The Rho family of G proteins is small G protein, which are involved in regulating actin cytoskeletal dynamics, cell movement, motility, transcription, cell survival, and cell growth. RHOA, RAC1, and CDC42 are the most studied proteins of the Rho family. Active G proteins are localized to the cellular membrane where they exert their maximal biological effectiveness.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “carrier” as used herein, refers to relatively nontoxic chemical compounds or agents that facilitate the incorporation of a compound into cells or tissues.

The term “axon” refers to projections of neurons that conduct signals to other cells through synapses. The term “axon growth” refers to the extension of the axon projection via the growth cone at the tip of the axon.

The term “neural disease” refers to diseases that compromise the cell viability of neurons. Neural diseases in which the etiology of said neural disease comprises formation of protein aggregates which are pathogenic to neurons provided that the protein aggregates are not related to the disease SBMA and are not intranuclear, include but are not limited to ALS, AD, Parkinson's Disease, multiple sclerosis, and prion diseases such as Kuru, Creutzfeltdt-Jakob disease, Fatal familial insomnia, and Gerstmann-Straussler-Scheinker syndrome. These neural diseases are also different from SBMA in that they do not contain polyglutamine repeats. Neural diseases can be recapitulated in vitro in tissue culture cells. For example, AD can be modeled in vitro by adding pre-aggregated 13-amyloid peptide to the cells. ALS can be modeled by depleting an ALS disease-related protein, TDP-43. Neural disease can also be modeled in vitro by creating protein aggregates through providing toxic stress to the cell. One way this can be achieved is by mixing dopamine with neurons such as neuroblastoma cells. These neural diseases can also be recapitulated in vivo in mouse models. A transgenic mouse that expresses a mutant Sodl protein has similar pathology to humans with ALS. Similarly, a transgenic mouse that overexpresses APP has similar pathology to humans with AD.

The term “pharmaceutically acceptable”, as used herein, refers to a material, including but not limited, to a salt, carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

The term “cyclodextrin,” as used herein, refers to cyclic carbohydrates consisting of at least six to eight sugar molecules in a ring formation. The outer part of the ring contains water soluble groups; at the center of the ring is a relatively nonpolar cavity able to accommodate small molecules.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. An appropriate “effective” amount in any individual case may be determined using techniques, such as a dose escalation study.

The term “patient”, “subject” or “individual” are used interchangeably. As used herein, they refer to individuals suffering from a disorder, and the like, encompasses mammals and non-mammals. None of the terms require that the individual be under the care and/or supervision of a medical professional. Mammals are any member of the Mammalian class, including but not limited to humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In some embodiments of the methods and compositions provided herein, the individual is a mammal. In preferred embodiments, the individual is a human.

The term “carrier” as used herein, refers to relatively nontoxic chemical compounds or agents that facilitate the incorporation of a compound into cells or tissues.

The term “about” when used before a numerical designation, e.g., temperature, time, amount, and concentration, including range, indicates approximations which may vary by (+) or (−) 10%, 5%, or 1%.

The term “halogenating” is defined as converting a hydroxy group to a halo group. The term “halo” or “halo group” refers to fluoro, chloro, bromo and iodo.

The term “stereoselectively” is defined as providing over 90% of the one geometric isomer for a newly formed double bond.

“Geometrical isomer” or “geometrical isomers” refer to compounds that differ in the geometry of one or more olefinic centers. “E” or “(E)” refers to the trans orientation and “Z” or “(Z)” refers to the cis orientation.

Geranylgeranyl acetone (GGA) refers to a compound of the formula V:

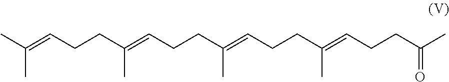 [SMILES: CC(=O)CC/C=C(\C)CC/C=C(\C)CC/C=C(\C)CCC=C(C)C]

wherein compositions comprising the compound are mixtures of geometrical isomers of the compound. The 5-trans isomer of geranylgeranyl acetone refers to a compound of the formula III:

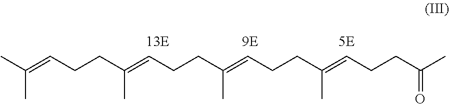 [SMILES: CC(=O)CC/C=C(\C)CC/C=C(\C)CC/C=C(\C)CCC=C(C)C]

wherein the number 5 carbon atom is in the 5-trans or 5E configuration. The 5-trans isomer also refers to (5E, 9E, 13E) geranylgeranyl acetone. The 5-cis isomer of geranylgeranyl acetone refers to a compound of the formula IV:

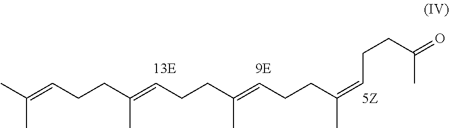 [SMILES: CC(=O)CC/C=C(/C)CC/C=C(\C)CC/C=C(\C)CCC=C(C)C]

wherein the number 5 carbon atom is in the 5-cis or 5Z configuration. The 5-cis isomer also refers to 5Z, 9E, 13E geranylgeranyl acetone.

### Compounds

Some embodiments of the present invention describe a pharmaceutical formulation comprising one or more isomers of a compound of formula I:

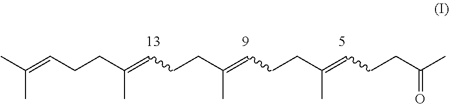 [SMILES: CC(=O)CCC=C(C)CCC=C(C)CCC=C(C)CCC=C(C)C]

in which the wavy line represents a bond having a configuration of the type (Z) or (E) or a mixture of the two configurations.

In some embodiments, geranylgeranyl acetone comprises a compound of formula II:

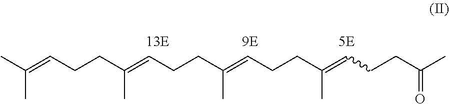 [SMILES: CC(=O)CCC=C(C)CC/C=C(\C)CC/C=C(\C)CCC=C(C)C]

in which the wavy line represents a bond having a configuration of the type (Z) or (E) or a mixture of the two configurations.

It will be clear to persons skilled in the art that in the compounds according to certain embodiments of the invention, the groups attached to the double bonds are fixed in different space as a result of the restricted rotation of double bonds. In some embodiments, provided herein is a compound of formula I or II, including all the stereoisomers, as well as mixtures thereof in any proportions, the Z and E isomers and mixtures thereof

Preferably in the compounds of formula I or II according to certain embodiments of the invention, the 5-alkene has the E configuration. In certain specific embodiments, the compound of formula I or II is the 5-trans isomer of GGA. In some embodiments, a compound of Formula I or II has the (5E, 9E, 13E) configuration. In some embodiments, the compound of formula I or II has the formula III:

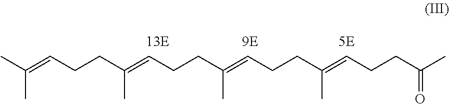 [SMILES: CC(=O)CC/C=C(\C)CC/C=C(\C)CC/C=C(\C)CCC=C(C)C]

In some embodiments, the compound of formula I, II or III is (5E, 9E, 13E) geranylgeranyl acetone. In some embodiments, the compound of formula I, II or III is in the form of a mixture of GGA isomers containing at least 80% by weight of the isomer having the (5E, 9E, 13E) configuration. In some embodiments, the compound of formula I, II or III is in the form of a mixture of GGA isomers containing at least 90% by weight of the isomer having the (5E, 9E, 13E) configuration. In some embodiments, the compound of formula I, II or III is in the form of a mixture of GGA isomers containing at least 85%, at least 90%, at least 95%, at least 96%, at least 97%, at least 98%, at least 99%, at least 99.5%, or at least 99.9% by weight of the isomer having the (5E, 9E, 13E) configuration. In other embodiments, the formulation comprises does not comprise a detectable amount of the 5-cis isomer of GGA. In other embodiments, the formulation comprises does not comprise a detectable amount of the GGA isomer of formula I or II having the 5Z, 9E, 13E configuration.

Other embodiments provided herein describe a pharmaceutical formulation comprising the 5-cis isomer of GGA. Some embodiments provided herein describe a pharmaceutical formulation comprising a compound of formula I or II wherein the 5-alkene has the Z configuration. In some embodiments, a compound of Formula I or II has the 5Z, 9E, 13E configuration. In some embodiments, the compound of formula I or II has the formula IV:

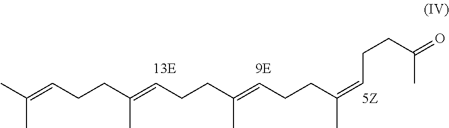 [SMILES: CC(=O)CC/C=C(/C)CC/C=C(\C)CC/C=C(\C)CCC=C(C)C]

In some embodiments, the compound of formula I, II or IV is (5E, 9E, 13E) geranylgeranyl acetone. In some embodiments, the compound of formula I, II, or IV in the form of a mixture of GGA isomers containing at least 80% by weight of the isomer having the (5E, 9E, 13E) configuration. In some embodiments, the compound of formula I, II or IV is in the form of a mixture of GGA isomers containing at least 80% by weight of the isomer having the 5Z, 9E, 13E configuration. In some embodiments, the compound of formula I, II or IV is in the form of a mixture of GGA isomers containing at least 85%, at least 90%, at least 95%, at least 96%, at least 97%, at least 98%, at least 99%, at least 99.5%, or at least 99.9% by weight of the isomer having the 5Z, 9E, 13E configuration. In some embodiments, the compound of formula I, II or IV is in the form of a mixture of GGA isomers containing at most 20%, at most 18%, at most 15%, at most 13%, at most 10%, at most 8%, at most 6%, at most 5%, at most 4%, at most 3%, at most 2%, at most 1%, or at most 0.5% by weight of the isomer having the 5Z, 9E, 13E configuration. In certain embodiments, the formulation comprises does not comprise a detectable amount of the 5-trans isomer of GGA. In other embodiments, the formulation comprises does not comprise a detectable amount of a compound of formula I, II or III having the (5E, 9E, 13E) configuration.

In some embodiments, any of the pharmaceutical formulations described herein comprise a compound of formula I, II, III, or IV, wherein the isomeric mixture of (5E, 9E, 13E) GGA to (5Z, 9E, 13E) GGA is in a ratio of about 50:50, 60:40, 75: 25, 80:20, 85:15, 90:10, 93:7, 95:5, 96:4, 97:3, 98:2, or 99:1. In some embodiments, (5E, 9E, 13E) geranylgeranyl acetone is present in a ratio of greater than 80:20 of (5E, 9E, 13E) to (5Z, 9E, 13E) geranylgeranyl acetone isomers. In some embodiments, (5E, 9E, 13E) geranylgeranyl acetone is present in a ratio of greater than 85:15 of (5E, 9E, 13E) to (5Z, 9E, 13E) geranylgeranyl acetone isomers. In some embodiments, (5E, 9E, 13E) geranylgeranyl acetone is present in a ratio of greater than 90:10 of (5E, 9E, 13E) to (5Z, 9E, 13E) geranylgeranyl acetone isomers. In some embodiments, (5E, 9E, 13E) geranylgeranyl acetone is present in a ratio of greater than 95:5 of (5E, 9E, 13E) to (5Z, 9E, 13E) geranylgeranyl acetone isomers. In some embodiments, (5E, 9E, 13E) geranylgeranyl acetone is present in a ratio of greater than 99:1 of (5E, 9E, 13E) to (5Z, 9E, 13E) geranylgeranyl acetone isomers.

The configuration of compounds is determined by methods known to those skilled in the art such as chiroptical spectroscopy and nuclear magnetic resonance spectroscopy.

A compound of formula I, II, III or IV may be synthesized according to the exemplary synthesis described below. For example, the compound of formula III is prepared following a method comprising one or more of the following steps:

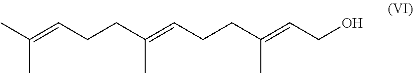 [SMILES: CC(C)=CCC/C(C)=C/CC/C(C)=C/CO]

(i) reacting a compound of formula VI under halogenation conditions to provide a compound of formula VII;

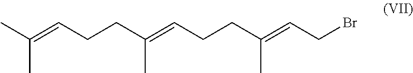 [SMILES: CC(C)=CCC/C(C)=C/CC/C(C)=C/CBr]

(ii) reacting the compound of formula VII with alkyl acetoacetate under alkylation conditions to provide a compound of formula VIII, where the stereochemistry at stereogenic center can be a racemic, R or S configuration:

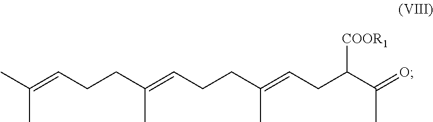

(iii) reacting the compound of formula VIII under hydrolysis and decarboxylation conditions to provide a compound of formula IX:

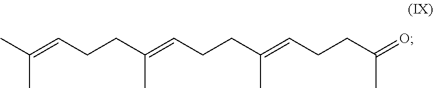 [SMILES: CC(=O)CC/C=C(\C)CC/C=C(\C)CCC=C(C)C]

(iv) reacting the compound of formula IX with a compound of formula X:

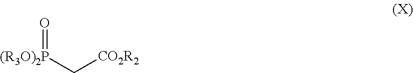

wherein R₂ and each R₃ independently are alkyl or substituted or unsubstituted aryl, under olefination conditions to selectively provide a compound of formula XI:

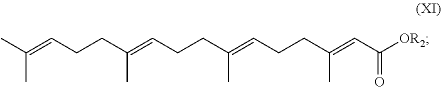

(v) reacting the compound of formula XI under reduction conditions to provide a compound of formula XII

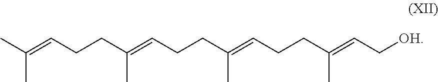 [SMILES: CC(C)=CCC/C(C)=C/CC/C(C)=C/CC/C(C)=C/CO]

Compound VI is combined with at least an equimolar amount of a halogenating agent typically in an inert solvent. As used in this application, an “inert solvent” is a solvent that does not react under the reaction conditions in which it is employed as a solvent. The reaction is typically run at a temperature of about 0° C. to 20° C. for a period of time sufficient to effect substantial completion of the reaction. Suitable solvents include, by way of example only, diethyl ether, acetonitrile, and the like. Suitable halogenating agents include PBr₃ or PPh₃/CBr₄. After reaction completion, the resulting product, compound IV, can be recovered under conventional conditions such as extraction, precipitation, filtration, chromatography, and the like or, alternatively, used in the next step of the reaction without purification and/or isolation.

Compound VII is combined with at least an equimolar amount of an alkyl acetoacetate, in the presence of a base and an inert solvent. The reaction is typically run initially at 0° C., and then warmed up to room temperature for a period of time sufficient to effect substantial completion of the reaction. Suitable solvents include, by way of example only, various alcohols, such as ethanol, dioxane, and mixtures thereof Suitable bases include, by way of example only, alkali metal alkoxides, such as sodium ethoxide.

Compound VIII is reacted with at least an equimolar amount, preferably, an excess of aqueous alkali. The reaction is typically run at about 40 to 80° C. and preferably about 80° C. for a period of time sufficient to effect substantial completion of the reaction. Suitable solvents include, by way of examples only, alcohols, such as methanol, ethanol, and the like.

Compound IX is combined with at least an equimolar amount, preferably, an excess of a compound of formula X, and at least an equimolar amount, preferably, an excess of base, in an inert solvent. The reaction is typically run, initially at about −30° C. for about 1-2 hours, and at room temperature for a period of time sufficient to effect substantial completion of the reaction. Suitable solvents include, by way of examples only, tetrahydrofuran, dioxane, and the like. Suitable bases include, by way of example only, alkali metal hydrides, such as sodium hydride, or potassium hexamethyldisilazide (KHMDS), or potassium tertiary butoxide (^{t}BuOK).

Compound XI is combined with a reducing agent in an inert solvent. The reaction is typically run at about 0° C. for about 15 minutes and at room temperature for a period of time sufficient to effect substantial completion of the reaction. Suitable reducing agents include, without limitation, LiAlH₄. Suitable solvents include, by way of examples only, diethyl ether, tetrahydrofuran, dioxane, and the like.

As will be apparent to the skilled artisan, after reaction completion, the resulting product can be recovered under conventional conditions such as precipitation, filtration, chromatography, and the like or, alternatively, used in the next step of the reaction without purification and/or isolation.

In some embodiments, the method further comprises repeating steps (i), (ii), and (iii) sequentially with a compound of formula XII to provide a compound of formula V.

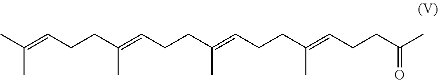 [SMILES: CC(=O)CC/C=C(\C)CC/C=C(\C)CC/C=C(\C)CCC=C(C)C]

In another embodiment, the synthetic method comprises repeating steps (i), (ii), (iii), (iv) and (v), sequentially, 1-3 times.

Also described herein is the synthetic method comprising one or more of the following steps:
(i) reacting a compound of formula XII:

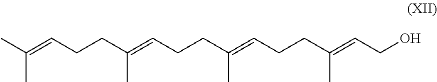 [SMILES: CC(C)=CCC/C(C)=C/CC/C(C)=C/CC/C(C)=C/CO]

under halogenation (e.g., bromination) condition to provide a compound of formula XIII

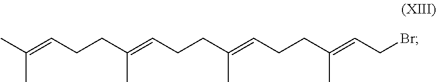 [SMILES: CC(C)=CCC/C(C)=C/CC/C(C)=C/CC/C(C)=C/CBr]

(ii) reacting the compound of formula XIII with alkyl acetoacetates, under alkylating conditions to provide a compound of formula XIV, where the stereochemistry at the stereogenic center is racemic or has an R or S configuration:

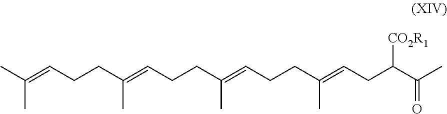

wherein R¹ alkyl is substituted or unsubstituted alkyl;
(iii) reacting a compound of formula XIV under hydrolysis and decarboxylation conditions to provide a compound of formula III:

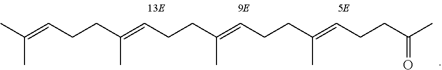 [SMILES: CC(=O)CC/C=C(\C)CC/C=C(\C)CC/C=C(\C)CCC=C(C)C]

(5E, 9E, 13E) or 5-trans GGA (III)

An exemplary synthesis of the compound of formula IV is described herein, the method of synthesis comprising step (i) or step (ii) or steps (i) and (ii):
(i) reacting a compound of formula XV:

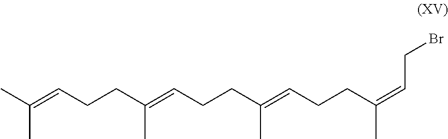 [SMILES: CC(C)=CCC/C(C)=C/CC/C(C)=C/CC/C(C)=C\CBr]

with alkyl acetoacetate under alkylating conditions to provide a compound of formula XVI, where the stereochemistry at the stereogenic center is racemic or has an R or S configuration:

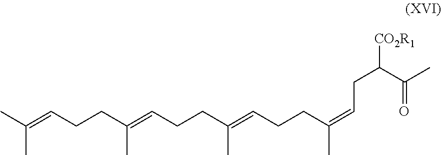

wherein R¹ alkyl is substituted or unsubstituted alkyl;
(ii) reacting a compound of formula XVI under hydrolysis and decarboxylation conditions to provide the compound of formula IV:

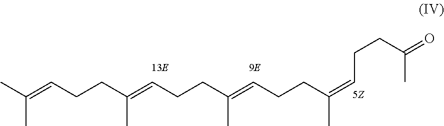 [SMILES: CC(=O)CC/C=C(/C)CC/C=C(\C)CC/C=C(\C)CCC=C(C)C]

In some embodiments, the compound of formula IV is synthesized by reacting a ketal compound of formula XVII:

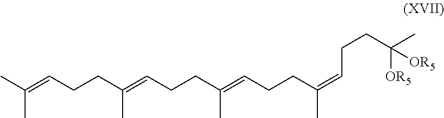

Wherein each R₅ independently is C₁-C₆ alkyl, or two R₅ groups together with the oxygen atoms they are attached to form a 5 or 6 membered ring, which ring is optionally substituted with 1-3, preferably 1-2, C₁-C₆ alkyl groups, under hydrolysis conditions to provide a compound of formula IV.

The ketal is combined with at least a catalytic amount, such as, 1-20 mol % of an aqueous acid, preferably, an aqueous mineral acid in an inert solvent. The reaction is typically run about 25° C. to about 80° C., for a period of time sufficient to effect substantial completion of the reaction. Suitable acids include, without limitation, HCl, H₂SO₄, and the like. Suitable solvents include alcohols, such as methanol, ethanol, tetrahydrofuran, and the like.

It will be apparent to the skilled artisan that the methods further employ routine steps of separation or purification to isolate the compounds, following methods such as chromatography (e.g., fractional distillation through a Fisher column), distillation (e.g., Kugelrohr distillation), or crystallization.

### Pharmaceutical Formulation

The compositions are comprised of in general, a compound of formula I, II, III, IV, GGA or a mixture thereof in combination with at least one pharmaceutically acceptable excipient. Some embodiments provided herein describe a pharmaceutical composition, wherein the composition further comprises one or more pharmaceutical carriers, excipients, auxiliaries, binders and/or diluents. Acceptable excipients are non-toxic, aid administration, and do not adversely affect the therapeutic benefit of the compound of this invention. Such excipients may be any solid, liquid, semi-solid. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Any composition described herein optionally comprises minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. In some embodiments, the composition further comprises one or more of lactose, dextrose, mannitol, pH buffering agents, antioxidant agents, preservative agents, tonicity adjusters or a combination thereof Examples of pharmaceutically acceptable carriers that are optionally used include, but are not limited to aqueous vehicles, nonaqueous vehicles, antimicrobial agents, local anesthetics, suspending and dispersing agents, emulsifying agents, sequestering or chelating agents and other pharmaceutically acceptable substances.

Aqueous suspensions contain the active material in admixture with excipients suitable for the manufacture of aqueous suspensions. Such excipients are suspending agents, for example sodium carboxymethylcellulose, methylcellulose, hydroxypropylmethyl-cellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose, saccharin or aspartame.

Suitable pharmaceutical carriers include inert diluents or fillers, water and various organic solvents.

In some embodiments, oily suspensions are formulated by suspending the active ingredient in a vegetable oil, for example arachis oil, olive oil, sesame oil or coconut oil, or in mineral oil such as liquid paraffin. In certain embodiments, the oily suspensions contain a thickening agent, for example beeswax, hard paraffin or cetyl alcohol. In further or additional embodiments, sweetening agents such as those set forth above, and flavoring agents are added to provide a palatable oral preparation. In other embodiments, these compositions are preserved by the addition of an anti-oxidant such as butylated hydroxyanisol or alpha-tocopherol.

In some embodiments, pharmaceutical compositions are in the form of oil-in-water emulsions. In some embodiments, the oily phase is a vegetable oil, for example olive oil or arachis oil, or a mineral oil, for example liquid paraffin or mixtures of these. Suitable emulsifying agents include but are not limited to naturally-occurring phosphatides, for example soy bean lecithin, and esters or partial esters derived from fatty acids and hexitol anhydrides, for example sorbitan monooleate, and condensation products of the said partial esters with ethylene oxide, for example polyoxyethylene sorbitan monooleate. In further or additional embodiments, the emulsions contain sweetening agents, flavoring agents, preservatives and antioxidants.

In some embodiments, the compounds or compositions described herein are delivered in a vesicle, such as a liposome. In further or alternative embodiments, the compounds and pharmaceutical compositions described herein are delivered in a controlled release system, or a controlled release system can be placed in proximity of the therapeutic target.

In certain embodiments, pharmaceutical compositions are administered via transdermal routes, using transdermal skin patches. To be administered in the form of a transdermal delivery system, the dosage administration will be continuous rather than intermittent throughout the dosage regimen.

Some embodiments provided herein describe a pharmaceutical formulation wherein the concentration of a compound of formula I, II, III, IV, GGA, or the trans-geranylgeranyl acetone isomer is from about 1% to about 99% by weight in the pharmaceutical compositions provided herein. In other embodiments, the concentration of any of the compounds described herein (e.g., geranylgeranyl acetone) is from about % to about 75%, or alternatively, from about 1% to about alternatively, from about 1% to about 30%, or alternatively, from about 1% to about 25%, or alternatively, from about % to about 20%, or alternatively, from about 2% to about 20%, or alternatively, from about 1% to about 10%, or alternatively, from about 10% to about 20%, or alternatively, from about 10% to about 15% by weight in the pharmaceutical composition. In certain embodiments, the concentration of any of the compounds (e.g., geranylgeranyl acetone) in the pharmaceutical composition is about 5% by weight, or alternatively, about 10%, or about 20%, or about 1%, or about 2%, or about 3%, or about 4%, or about 6%, or about 7%, or about 8%, or about 9%, or about 11%, or about 12%, or about 14%, or about 16%, or about 18%, or about 22%, or about 25%, or about 26%, or about 28%, or about 30%, or about 32%, or about 34%, or about 36%, or about 38%, or about 40%, or about 42%, or about 44%, or about 46%, or about 48%, or about 50%, or about 52%, or about 54%, or about 56%, or about 58%, or about 60%, or about 64%, or about 68%, or about 72%, or about 76%, or about 80% by weight.

An effective amount of 5-trans GGA is the amount of GGA required to produce a protective effect in vitro or in vivo. In some embodiments the effective amount in vitro is about from 0.1 nM to about 1 mM. In some embodiments the effective amount in vitro is from about 0.1 nM to about 0.5 nM or from about 0.5 nM to about 1.0 nM or from about 1.0 nM to about 5.0 nM or from about 5.0 nM to about 10 nM or from about 10 nM to about 50 nM or from about 50 nM to about 100 nM or from about 100 nM to about 500 nM or from about 500 nM to about 1 mM. In some embodiments, the effective amount for an effect in vivo is about 0.1 mg to about 100 mg, or preferably, from about 1 mg to about 50 mg, or more preferably, from about 1 mg to about 25 mg per kg/day, or from about 1 mg to about 12 mg per kg/day. In some other embodiments, the effective amount in vivo is from about 10 mg/kg/day to about 100 mg/kg/day, about 20 mg/kg/day to about 90 mg/kg/day, about 30 mg/kg/day to about 80 mg/kg/day, about 40 mg/kg/day to about 70 mg/kg/day, or about 50 mg/kg/day to about 60 mg/kg/day. In some embodiments, the effective amount in vivo is from about 1 mg/kg/day to about 5 mg/kg/day, In some embodiments, the effective amount in vivo is from about 6 mg/kg/day to about 12 mg/kg/day, In one embodiment, the effective amount in vivo is about 3 mg/kg/day. In another embodiment, the effective amount in vivo is about 6 mg/kg/day. In another embodiment, the effective amount in vivo is about 12 mg/kg/day. In still some other embodiments, the effective amount in vivo is from about 100 mg/kg/day to about 1000 mg/kg/day.

Compounds and pharmaceutical formulations of this invention may be used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

### Transdermal Systems or Patches

Some embodiments described herein relate to transdermal systems or patches that deliver therapeutically effective amount of drug to the systemic circulation via skin. In some embodiments, drugs delivered through transdermal patches have unique advantages. These advantages include but are not limited to avoiding first pass metabolism of drug(s), avoiding irritation of the GI mucosa, avoiding fluctuation in drug levels, predictable and extended duration of activity, minimizing undesirable side effects, suitability for drugs with short half-life and narrow therapeutic index, maintaining steady plasma concentrations of potent drugs, greater patient compliance due to elimination of multiple doses and dosage forms (oral and systemic), suitability for self-administration, and ease of terminating of therapy at any point in time.

Provided in some embodiments is medical patch or device for the rate-controlled transdermal administration of any of the compounds described herein through intact skin for an extended period of time which comprises: (A) a drug reservoir comprising any compound described herein and (b) a drug-impermeable backing. In some embodiments, the pharmaceutical patch optionally comprises a skin permeation enhancer. In some instances, the patch allow for treating neural disorders, which comprises administering a compound of formula I, II, III, or IV through an area of intact skin at a therapeutically effective rate for an extended period of time with a medical patch as described herein.

Provided herein in some embodiments, is a matrix type transdermal system where the drug reservoir is prepared by dissolving the drug and polymer in a common solvent. In some embodiments, an insoluble drug is homogeneously dispersed in hydrophilic polymer. In other embodiments, an insoluble drug is homogeneously dispersed in lipophilic polymer. In further or additional embodiments, the matrix type transdermal system optionally contains one or more suitable plasticizer and/or permeation enhancer. In some embodiments, the drug is loaded on a reservoir type transdermal patch. In other embodiments, the drug is loaded on a polymer matrix.

Other embodiments provided herein describe a reservoir transdermal system, wherein the drug reservoir is made of a homogeneous dispersion of drug particles suspended in an unleachable viscous liquid medium. In certain embodiments, the dispersion forms a paste-like suspension, gel or clear solution comprising the drug in a releasable solvent. In some embodiments, the reservoir is sandwiched between a rate controlling membrane and backing laminate.

Also provided herein in some embodiments is a membrane matrix hybrid transdermal system or patch. In some embodiments, the membrane matrix hybrid system is a modified reservoir type transdermal system. In certain embodiments, the liquid formulation of the drug reservoir is replaced with a solid polymer matrix. In further or additional embodiments, the solid polymer matrix is sandwiched between a rate controlling membrane and backing laminate.

Some embodiments provided herein describe a microreservoir system, wherein the drug reservoir is formed by suspending the drug particles in a solution. In some embodiments, the solution is an aqueous solution. In further or additional embodiments, the aqueous solution further comprises a water miscible drug solubilizer. In some embodiments, the drug suspension is homogeneously dispersed by a high shear mechanical force in lipophilic polymer, forming thousands of unleachable microscopic drug reservoirs, or microreservoirs. In some embodiments, the adhesive layer is applied along the circumference of the patch to form a strip of adhesive rim surrounding the drug reservoir. In some instances, the release of drug follows either a partition-controlled or matrix diffusion-controlled process depending on the solubilities of the drug in the liquid compartments and in the polymer matrix.

Other embodiments in provided herein describe drug in adhesive type systems. In some embodiments, the drug and other formulation components are directly incorporated into an organic solvent based pressure sensitive adhesive solution, mixed and casted as a thin film. In some embodiment, an adhesive matrix film containing the drug and excipients is formed. In some embodiments, the drug in adhesive matrix is sandwiched between release liner and backing layer. In some embodiments, the transdermal patch contains a single layer. In other embodiments, the transdermal patch contains a double layer. In other embodiments, the transdermal patch contains three layers. In further embodiments, the transdermal patch contains multiple layers.

In certain embodiments, the drug in adhesive type system is a polymer matrix gradient-controlled patch, wherein the drug reservoir comprises multi-laminate adhesive layers of a pressure-sensitive adhesive polymer wherein the drug and any optional excipients and/or permeation enhancers are dispersed in a proportional manner, forming a concentration gradient which increases from the skin contacting surface toward the drug-impermeable layer.

Polymer Matrix/Drug Reservoir

Some embodiments provided herein describe a pharmaceutical transdermal formulation, wherein the drug reservoir exists in solid, suspension or solution form. In some embodiments, the drug reservoir comprises a solid polymer matrix. In some embodiments, the drug is loaded onto a polymer matrix by dispersion or extrusion methods. In further embodiments, the drug formulation is introduced to the reservoir by injection molding, spray coating, microencapsulation or other techniques known in the art.

In some embodiments, the polymers control the release of the drug from the device. In some embodiments, the release of drug is controlled by approximately choosing the loading dose and the polymer. Examples of polymers used to form the matrix or reservoir include but are not limited to amidated pectin, carbomer, carboxy methyl cellulose, carboxy methyl cellulose sodium, cellulose acetate, copovidone, crosslinked polyethylene glycol, ethyl cellulose, eudragit E-100, eudragit NE-40D, eudragit RL 100, eudragit RL PM, eudragit RS 100, eudragit RS PM, eudragit S-100, guar gum, hydroxypropyl cellulose, hydroxypropylmethyl cellulose, methyl cellulose, pluronic lecithin organogels, polycarbophil, polymethacrylates, polyvinylalcohol, polyvinylpyrrolidone, sodium alginate, and xanthan gum.

Rate-Controlling Membrane

In some embodiments described herein, transdermal patch contains an inert membrane for enclosing the drug. In certain embodiments, the inert membrane controls the rate of diffusion of the drug. Examples of polymers used to form these inert membranes include but are not limited to ammonium alginate, bentonite, chitosan, collagen, copovidone, dibutyl phthalate, diethyl phthalate, dimethyl phthalate, ethyl lactate, ethylene vinyl acetate, gelatin, hydroxypropyl cellulose, hydroxypropylmethyl cellulose, hypromellose acetate succinate, poly(methylvinyl ether/maleic anhydride) copolymers, poly-2-hydroxyethyl methacrylate, polymethacrylates, polyurethane, shellac, and silicon rubber.

In some embodiments, the rate-controlling membrane is from about 0.5-5 mm thick and preferably about 1-3 mm thick. In specific embodiments, the rate-controlling membrane comprises ethylene-vinyl acetate copolymer, a poly vinyl acetate or polypropylene. In some instances, the loading is from about 5-50 mg/cm² yielding a dry loading of from about 0.01-5 mg/cm².

Permeation Enhancers

Any transdermal formulation described herein optionally comprises permeation enhancers. In some embodiments, the permeation enhances alter the protein and/or lipid structure of the stratum corneum and thereby, increase the permeability of drugs. In some instances, skin permeation enhances induce a temporary, reversible increase in skin permeability. Examples of transdermal permeation enhancers include but are not limited to fatty acids, monoglyceride esters of such fatty acids, C₈₋₁₈ alkylsaccharides (e.g., n-octyl-beta-D-flucopyranoside, n-lauryl-beta-D-glycopyranoside, etc.), C₈₋₁₈ acyl carnitines, C₁₋₁₄ alkyl methylsufloxides (e g , dimethylsulfoxide, decyl emthylsulfoxide, etc.), 1-(N,N-dimethylamino)-2-propanol decanoate), 1-butyl pyrrolidone, 1-ethylpyrrolidone, 2-n-nonyl-1, 3-dioxolane (SEPA), 2-pyrrolidone, arachidonic acid, anethole, Brij 32,50,58,72,97,700, caprylic acid, capric acid, caraway oil, cardamom oil, carvacrol, cineole, crovol A40, crovol PK40, decyl alcohol, diethanolamine, dimethyl formamide, dimethylacetamide, dimethylsulfoxide (DMSO), dipentene, d-limonene, dodecyl alcohol, dodecylamine, ethanol, ethyl acetate, ethoxylated triglycerides, ethyldecanoate, ethyloleate, glyceryl monocaprylate, glyceryl monolaurate, glyceryl monooleate, hydroxypropyl β-cyclodextrin, isopropyl myristate, isopropyl palmitate, labrafac, labrafil, labrafil 1944, lauric acid, laurocapram (azone or 1-dodecylazacycloheptan-2-one), lauroglycol, lauryl alcohol, lemon oil, linoleic acid, linolenic acid, menthol, methanol, mineral oil, myristic acid, myristyl alcohol, N,N-dimethylaminoacetate, N-decylmethyl sulfoxide, N-methyl pyrrolidone, oleic acid, oleyl alcohol, palmitic acid, palmitoleic acid, phenethylamine, plurol olieque CC497, pluronic, p-menthane, polyethylene glycol, polyethyleneglycol oleamine, polyoxyethylene castor oil derivatives, polyoxyethylene ethers, propylene glycol, sodium lauryl sulfate (SLS), sorbitan monolaurate, stearic acid, stearyl alcohol, stearylamine, thymol, transcutol, tricaprylin, triethylamine, tween 80, andβ-cyclodextrin.

Permeation enhancers are used in an amount that does not damage the skin of the subject to be treated. In some embodiments, the weight-by-weight ratio (drug):(permeation enhancer) in the patch ranges from 10:1 to 10:1. In some embodiments, the weight-by-weight ratio (drug):(permeation enhancer) in the patch ranges from about 1:1 to about 1:5.

Pressure Sensitive Adhesives (PSA)

In some embodiments described herein, a transdermal patch contains one or more pressure sensitive adhesive (PSA). In some embodiments, the PSA maintains an intimate contact between the transdermal patch and the skin surface by application of gentle pressure. Non-limiting examples of pressure sensitive adhesives include isopropyl myristate, methacrylamide, methacrylic acid, N-alkoxyalkyl or N-alkyl-acrylamides, poly(ethylene/butylene)-polystylene, poly(ethylene/propylene)-polystylene, polyacrylates, polybutadiene, polyisobutylene, polyisoprene, polystyrene, silicon based adhesives, and styrene-butadiene. In some embodiments, the adhesive layer comprises a drug-compatible, hypoallergenic pressure-sensitive adhesive polymer. In certain embodiments, a peripheral ring of adhesive is outside the path of drug from the system to the skin. In such instances, the adhesive does not need to be drug-compatible.

Backing Laminates

Some embodiments provided herein describe pharmaceutical transdermal patch wherein the patch further comprises one or more backing laminate. In some embodiments, backing laminates provide support to the transdermal patches. In certain embodiments, these laminates have high flexibility, good oxygen transmission and/or high moisture vapor transmission rate. Examples of backing laminates used in transdermal patches include but are not limited to aluminum vapor coated layer and polyester films, ethylcellulose, ethylene vinyl acetate, polyethylene, polyvinyl chloride. In some embodiments, is a plastic laminate (e.g., a polyester film laminate or a polyester-polyurethane film). In further or additional embodiments, the backing is drug-impermeable.

Release Liner

In some embodiments, a transdermal formulation described herein comprises a release liner. In some embodiments, release liners protect the patch until it is applied onto the skin. Examples of release liners used in transdermal patches include non-occlusive (paper fabric), occlusive (polyethylene, polyvinylchloride), silicon or teflon, polyester foil and metallized laminates.

Solvents

Any transdermal formulation described herein optionally comprises one or more solvents. In some embodiments, solvents are utilized to prepare the drug reservoir. Non-limiting examples of solvents used in transdermal patches include acetone, butanol, butylene glycol, chloroform, dichloromethane, diethyl ether, ethanol, ethyl acetate, ethyl methyl ketone, ethyl oleate, isopropanol, lauryl alcohol, linolenyl alcohol, methanol, octyldodecanol, 1-propanol, propylene glycol, sesame oil, and tetrahydrofuran.

Plasticizers

Some embodiments provided herein describe pharmaceutical transdermal patch wherein the patch further comprises one or more plasticizers. In some embodiments, plasticizers improve the flexibility or plasticity of transdermal patches. Examples of plasticizers include but are not limited to acetyltributyl citrate, acetyltriethyl citrate, benzyl benzoate, chlorobutanol, dibutyl phthalate, dibutyl sebacate, diethyl phthalate, dimethyl phthalate, isopropyl myristate, glycerin, mannitol, mineral oil and lanolin alcohols, petrolatum and lanolin alcohols, polyethylene glycol, propylene glycol, sorbitol, triacetin, tributyl citrate, and triethyl citrate.

Other Additives

Any suitable drug reservoir described above optionally comprises further ingredients. In order to prevent the growth of micro-organisms such as bacteria, yeasts and fungi in the patches, a preservative agent is optionally added. Suitable preservatives include but are not limited to benzoic acid, sorbic acid, methylparaben, propylparaben, imidazolidinyl urea (=Germall 115®) and diazolidinyl urea (=Germall II®), phenoxetol, benzyl alcohol, quaternary compounds, e.g. benzylalkonium chloride, and the like. The concentration of the preservatives may range from 0.05% to 1%, particularly from 0.1% to 0.5%, and most particularly is about 0.2%.

The drug reservoir may also be sterilized following art-known procedures. Drug reservoirs may be sterilized by irradiation with gamma rays. Drug solutions can be filtered aseptically and then sterilized by autoclaving.

In some embodiments, the patches optionally include stabilizers (EDTA), antioxidants (BHT, BHA), viscosity regulating agents, surfactants (especially non-ionic), hydrating agents (urea), and/or the like ingredients.

In one embodiment, this invention provides sustained release formulations such as drug depots or patches comprising an effective amount of a compound of formula I, II, III, or IV (e.g., 5E, 9E, 13E geranylgeranyl acetone). In another embodiment, the patch further comprises gum arabic or hydroxypropyl cellulose separately or in combination, in the presence of alpha-tocopherol. Preferably, the hydroxypropyl cellulose has an average MW of from 10,000 to 100,000. In another embodiment, the hydroxypropyl cellulose has an average MW of from 5,000 to 50,000. The patch contains, in various embodiments, an amount of GGA, preferably the (5E, 9E, 13E) isomer of it, which is sufficient to maintain a therapeutically effective amount GGA in the plasma for about 12 hours. In one embodiment, the GGA comprises at least 80%, at least 85%, at least 90%, at least 95%, or at least 99% of the (5E, 9E, 13E) isomer of GGA.

Transdermal patches are tested for various physicochemical, in vitro, and in vivo properties. The thickness of the drug loaded patch is determined using microscope, dial gauge, screw gauge or micrometer. Usually, measurements are made at different points. Weight variation is studied individually by weighing 10 randomly selected patches and calculating the average weight. In some instances, moisture content of transdermal patches is determined by weight loss on drying method, or Karl Fisher titrations. In some instances, moisture uptake by transdermal patches is measured by exposing the patches to higher humidity conditions, and determining any weight increase after a fixed time point. Flatness of the transdermal patches is determined as follow: (i) one strip is cut from the centre, and two from each side of patches; (ii) the length of each strip is measured and variation in length is measured by determining percent constriction. Zero percent constriction is considered as 100 percent flatness.

Folding endurance is determined by repeatedly folding the film at the same place until it breaks. The number of times the patch could be folded at the same place without breaking is folding endurance value.

To determine tensile strength, one end of the films is kept fixed with the help of an iron screen and other end is connected to a freely movable thread over a pulley. Weights are added gradually to the pan attached with the hanging end of the thread. A pointer on the thread is used to measure the elongation of the film. The weight just sufficient to break the film is noted. The tensile strength is calculated using the following equation: Tensile strength=F/a.b (1+L/l); where, F is the force required to break; a is width of film; b is thickness of film; L is length of film; 1 is elongation of film at break point.

Also provided herein is a study to determine water vapor transmission (WVT). In some instances, one gram of calcium chloride is placed in previously dried empty vials having equal diameter. The polymer films are pasted over the brim with the help of adhesive like silicon adhesive grease and the adhesive is allowed to set for 5 minutes. The vials are accurately weighed and placed in humidity chamber maintained at 68% RH. Any increase in the weight of vials after 1^{st} day, 2^{nd} day, 3rd day up to 7 consecutive days is considered as a quantitative measure of moisture transmitted through the patch. In some instances, the WVT is calculated using the following equation: WVT=W/ST; where, W is the increase in weight at different time interval; S is area of film exposed (cm²); T is exposure time.

Distribution of drug and polymer in the film is studied using scanning electron microscope. Sections of each sample are cut and then mounted onto stubs using double sided adhesive tape. The sections are then coated with gold palladium alloy using fine coat ion sputter to render them electrically conductive. Then the sections are examined under scanning electron microscope.

The peel adhesion properties of a transdermal patch are tested by measuring the force required to pull a single coated tape, applied to substrate at 180° angle. The test is passed if there is no residue on the substrate.

Tack properties include the ability of the polymer to adhere to substrate with little contact pressure. Tack is dependent on molecular weight and composition of polymer as well as on the use of tackifying resins in polymer. It is measured by different approaches such as thumb tack test, rolling ball method, quick stick method, and probe tack methods. The force required to remove thumb from adhesive is a measured in Thumb tack method. Rolling ball method involves measurement of the distance that stainless steel ball travels along an upward facing adhesive. The less tacky the adhesive, the further the ball will travel. In quick stick (peel tack) method, the peel force required to break the bond between an adhesive and substrate is measured by pulling the tape away from the substrate at 90° at the speed of 12 inch/min. Probe tack test measures the force required to pull a probe away from an adhesive at a fixed rate.

Shear strength properties or creep resistance are measured to determine the cohesive strength of an adhesive polymer, determined by measuring the time it takes to pull an adhesive coated tape off a stainless plate.

Drug content is determined by dissolving an accurately weighed portion of the film in a suitable solvent of specific volume in which drug is soluble and the filtered solution is analyzed by a suitable analytical method (UV, HPLC).

Content uniformity is also determined In some instances, about 10 patches are selected, and content is determined for individual patches by a by a suitable analytical method (UV, reflection index, HPLC).

A suitable patch according to the present invention delivers a compound of formula I, II, III or IV (e.g., 5E, 9E, 13E geranylgeranyl acetone) through about 5-100 cm², about 10-50 cm², or about 20 cm² of intact skin over an extended period of time, and at a rate within the range of about 0.5 to 20 μg/cm².h or at a rate within the range of approximately 1-5 μg/cm².h. When so delivered it is possible, by appropriate selection of the surface area of the drug delivery device to obtain total drug input rates which provide an adequate range of titration for individual patient needs while maintaining a safe and effective dosage form. A suitable patch according to embodiments described herein comprises sufficient compound and optional permutation enhancers to allow administration for up to 10 days, 9 days, 8 days, 7 days, 6 days, 5 days, 4 days, 3 days, 2 days, 1 days, 18 hours, 12 hours, 10 hours or 6 hours.

In some embodiments, the transdermal patch or system delivers controlled amounts of compound to patients for extended period of time ranging in duration from several hours to several days. In some embodiments, the totally daily dose of GGA ranges from about 5 to about 500 mg, about 5 to about 250 mg, about 5 to about 100 mg, about 5 to about 75 mg, about 100 to about 500 mg, about 100 to about 250 mg, about 25 to about 250 mg, about 25 to about 100 mg, about 25 to about 75 mg, or from about 30 to 60 mg. In some embodiments, the totally daily dose of GGA ranges from about 2 to about 8 mg or from about 0.5 to 2 mg. In some instances, the transdermal patch provides for administration of compound at a continuous controlled rate and avoids excessive peak levels. In some embodiments, negative side effects are alleviated with transdermal administration. In some instances, the device delivers the drug a substantially constant rate for at least about 24 hours while at the same time keeping the amount of drug within both the unused and depleted systems to a minimum In some embodiments, the system itself controls the maximum rate at which the drug is delivered through the skin 1001161A compound of formula I, II, III, or IV (e.g., 5E, 9E, 13E geranylgeranyl acetone) in the presence of a permeation enhancer is administered to the human body via the transdermal route at a therapeutically effective rate for an extended period of time. In some embodiments, the rate of administration is in the range of 10 to 400 μg/h for a substantial portion of the extended period of time. In some instances, steady-state administration rates are from about 10-300 μg/h or from about 25-150 μg/h.

### Methods of Treatment

Some embodiments provided herein describe a method of treating a neural disease. In some instances, neural diseases are characterized by neuroinflammation. Examples of such neural diseases include, but are not limited to, amyotrophic lateral sclerosis (ALS), Alzheimer's disease, Parkinson's disease, multiple sclerosis, prion diseases such as Kuru, Creutzfeltdt-Jakob disease, fatal familial insomnia, Gerstmann-Straussler-Scheinker syndrome, and damage to the spinal cord. Also provided herein in some embodiments is a method of treating visual disorders such as optic neuropathy, glaucoma, degeneration of optic nerves, age-related macular degeneration (AMD) and ophthalmoplegia. Some embodiments described herein provide a pharmaceutical formulation for preventing neural death during epileptic seizures. Any pharmaceutical formulation and/or compounds described above are useful in the methods described herein.

Provided herein, in some embodiments, are methods for using effective amounts of one or more compounds of formula I, II, III or IV, preferably having the (5E, 9E, 13E) configuration or the, optionally with at least one pharmaceutically acceptable excipient for inhibiting neural death and/or increasing neural activity. In some embodiments, the compound formula I, II, III or IV is the trans-GGA or the synthetic trans-GGA. For example, and without limitation, methods provided here in describe impeding the progression of neural diseases or injury using one or more compounds of formula I, II, III or IV.

In one aspect, methods for increasing the axon growth of neurons by contacting said neurons with the pharmaceutical compositions are provided herein. In some cases, neural diseases result in an impairment of signaling between neurons. In some cases, this impairment is due in part to a reduction in the growth of axonal projections. In some embodiments, contacting neurons with a compound of formula I, II, III, IV, or GGA enhances axonal growth. In some embodiments, a compound of formula I, II, III, IV, or GGA restores axonal grown in neurons afflicted with a neural disease. In a related embodiment, the pre-contacted neurons exhibit a reduction in the axon growth ability.

One embodiment provided herein describes a method for inhibiting the cell death of neurons susceptible to neuronal cell death, which method comprises contacting said neurons with the pharmaceutical compositions provided herein. Neurons susceptible to neuronal cell death include those that have the characteristics of a neural disease and/or those that have undergone injury or toxic stress. One method of creating toxic stress to a cell is by mixing dopamine with neurons such as neuroblastoma cells. Another source of toxic stress is oxidative stress. Oxidative stress can occur from neuronal disease or injury. It is contemplated that contacting neurons with a compound of formula I, II, III, IV, or GGA will inhibit their death as measured by a MTT assay or other techniques commonly known to one skilled in the art.

In another aspect, there are methods for increasing the neurite growth of neurons by contacting said neurons with the pharmaceutical compositions provided herein. The term “neurite” refers to both axons and dendrites. Neural diseases can result in an impairment of signaling between neurons. In some cases, this impairment is due in part to a reduction in the growth of axonal and/or dendritic projections. It is contemplated that contacting neurons with a compound of formula I, II, III, IV, or GGA will enhance neurite growth. It is further contemplated that a compound of formula I, II, III, IV, or GGA will restore neurite grown in neurons afflicted with a neural disease. In a related embodiment, the pre-contacted neurons exhibit a reduction in the neurite growth ability.

One embodiment of this invention is directed to a method for increasing the expression and/or release of one or more neurotransmitters from a neuron by contacting said neurons with the pharmaceutical compositions provided herein. It is contemplated that contacting neurons with an effective amount of a compound of formula I, II, III, IV, or GGA will increase the expression level of one or more neurotransmitters. It is also contemplated that contacting neurons with a compound of formula I, II, III, IV, or GGA will increase the release of one or more neurotransmitters from neurons. The release of one or more neurotransmitters refers to the exocytotic process by which secretory vesicles containing one or more neurotransmitters are fused to cell membrane, which directs the neurotransmitters out of the neuron. It is contemplated that the increase in the expression and/or release of neurotransmitters will lead to enhanced signaling in neurons, in which levels of expression or release of neurotransmitters are otherwise reduced due to the disease. The increase in their expression and release can be measured by molecular techniques commonly known to one skilled in the art.

One embodiment of this invention is directed to a method for inducing synapse formation of a neuron by contacting said neurons with the pharmaceutical compositions provided herein. A synapse is a junction between two neurons. Synapses are essential to neural function and permit transmission of signals from one neuron to the next. Thus, an increase in the neural synapses will lead to an increase in the signaling between two or more neurons. It is contemplated that contacting the neurons with an effective amount of a compound of formula I, II, III, IV, or GGA will increase synapse formation in neurons that otherwise experience reduced synapse formation as a result of neural disease.

Another embodiment of this invention is directed to a method for increasing electrical excitability of a neuron by contacting said neurons with the pharmaceutical compositions provided herein. Electrical excitation is one mode of communication among two or more neurons. It is contemplated that contacting neurons with an effective amount of a compound of formula I, II, III, IV, or GGA will increase the electrical excitability of neurons in which electrical excitability and other modes of neural communication are otherwise impaired due to neural disease. Electrical excitability can be measured by electrophysiological methods commonly known to one skilled in the art.

In each of the three previous paragraphs above, the administration of a compound of formula I, II, III, IV, or GGA enhances communication between neurons and accordingly provides for a method of inhibiting the loss of cognitive abilities in a mammal that is at risk of dementia or suffering from incipient or partial dementia while retaining some cognitive skills. Incipient or partial dementia in a mammal is one in which the mammal still exhibits some cognitive skills, but the skills are being lost and/or diminished over time. Method comprises administering an effective amount of a compound of formula I, II, III, IV, or GGA to said patient.

In another embodiment, this invention is directed to a method for inhibiting the death of neurons due to formation of or further formation of pathogenic protein aggregates between, outside or inside neurons, wherein said method comprises contacting said neurons at risk of developing said pathogenic protein aggregates with the pharmaceutical compositions provided herein, provided that said pathogenic protein aggregates are not related to SBMA. In one embodiment of this invention, the pathogenic protein aggregates form between or outside of the neurons. In another embodiment of this invention, the pathogenic protein aggregates form inside said neurons. In one embodiment of this invention, the pathogenic protein aggregates are a result of toxic stress to the cell. One method of creating toxic stress to a cell is by mixing dopamine with neurons such as neuroblastoma cells. It is contemplated that contacting neurons with a compound of formula I, II, III, IV, or GGA will inhibit their death as measured by a MTT assay or other techniques commonly known to one skilled in the art.

Another embodiment of the invention is directed to a method for protecting neurons from pathogenic extracellular protein aggregates which method comprises contacting said neurons and/or said pathogenic protein aggregates with the pharmaceutical compositions provided herein. In one embodiment of this invention, contacting said neurons and/or said pathogenic protein aggregates with the pharmaceutical compositions provided herein. Without being limited to any theory, it is contemplated that contacting the neurons and/or the pathogenic protein aggregates with a compound of formula I, II, III, IV, or GGA will solubilize at least a portion of the pathogenic protein aggregates residing between, outside, or inside of the cells. It is further contemplated that contacting the neurons and/or the pathogenic protein aggregates with a compound of formula I, II, III, IV, or GGA will alter the pathogenic protein aggregates in such a way that they are non-pathogenic. A non-pathogenic form of the protein aggregate is one that does not contribute to the death or loss of functionality of the neuron. There are many assays known to one skilled in the art for measuring the protection of neurons either in cell culture or in a mammal. One example is a measure of increased cell viability by a MTT assay. Another example is by immunostaining neurons in vitro or in vivo for cell death-indicating molecules such as, for example, caspases or propidium iodide.

In yet another embodiment of the invention is directed to a method for protecting neurons from pathogenic intracellular protein aggregates which method comprises contacting said neurons with the pharmaceutical compositions provided herein provided that said protein aggregation is not related to SBMA. This method is not intended to inhibit or reduce negative effects of neural diseases in which the pathogenic protein aggregates are intranuclear or diseases in which the protein aggregation is related to SBMA. SBMA is a disease caused by pathogenic androgen receptor protein accumulation. It is distinct from the neural diseases mentioned in this application since the pathogenic protein aggregates of SBMA contain polyglutamines and are formed intranuclearly. It is also distinct from the neural diseases described in this application because the protein aggregates are formed from androgen receptor protein accumulation. It is contemplated that contacting neurons with an effective amount of a compound of formula I, II, III, IV, or GGA will alter the pathogenic protein aggregate into a non-pathogenic form.

One embodiment of the invention is directed to a method of modulating the activity of G proteins in neurons which method comprises contacting said neurons with the pharmaceutical compositions provided herein. It is contemplated that contacting neurons with a compound of formula I, II, III, IV, or GGA will alter the sub-cellular localization, thus changing the activities of the G protein in the cell. In one embodiment of the invention, contacting neurons with a compound of formula I, II, III, IV, or GGA will enhance the activity of G proteins in neurons. It is contemplated that contacting a compound of formula I, II, III, IV, or GGA with neurons will increase the expression level of G proteins. It is also contemplated that contacting a compound of formula I, II, III, IV, or GGA with neurons will enhance the activity of G proteins by changing their sub-cellular localization to the cell membranes where they must be to exert their biological activities.

One embodiment of the invention is directed to a method of modulating or enhancing the activity of G proteins in neurons at risk of death which method comprises contacting said neurons with the pharmaceutical compositions provided herein. Neurons may be at risk of death as a result of genetic changes related to ALS. One such genetic mutation is a depletion of the TDP-43 protein. It is contemplated that neurons with depleted TDP-43 or other genetic mutations associated with ALS will have an increase or change in the activity of G proteins after being contacted with a compound of formula I, II, III, IV, or GGA. It is further contemplated that a compound of formula I, II, III, IV, or GGA will result in an increase in the activity of G proteins in these cells by changing their sub-cellular localization to the cell membranes where they must be to exert their biological activities.

Another embodiment of the invention is directed to a method for inhibiting the neurotoxicity of β-amyloid peptide by contacting the β-amyloid peptide with the pharmaceutical compositions provided herein. In one embodiment of the invention the β-amyloid peptide is between or outside of neurons. In yet another embodiment of the invention, the β-amyloid peptide is part of the β-amyloid plaque. It is contemplated that contacting neurons with a compound of formula I, II, III, IV, or GGA will result in solubilizing at least a portion of the β-amyloid peptide, thus decreasing its neurotoxicity. It is further contemplated that a compound of formula I, II, III, IV, or GGA will decrease the toxicity of the β-amyloid peptide by altering it in such a way that it is no longer toxic to the cell. It is also believed that a compound of formula I, II, III, IV, or GGA will induce the expression of heat shock proteins (HSPs) in the neurons. It is also contemplated that HSPs will be induced in support cells such as glial cells. The induced heat shock proteins in the neurons or glial cells may be transmitted extracellularly and act to dissolve extracellular protein aggregates. Cell viability can be measured by standard assays known to those skilled in the art. One such example of an assay to measure cell viability is a MTT assay. Another example is a MTS assay. The modulation of protein aggregation can be visualized by immunostaining or histological staining techniques commonly known to one skilled in the art.

One embodiment of the invention is directed to a method for inhibiting neural death and increasing neural activity in a mammal suffering from neural diseases, wherein the etiology of said neural diseases comprises formation of protein aggregates which are pathogenic to neurons, and which method comprises administering to said mammal the pharmaceutical compositions provided herein. This method is not intended to inhibit neural death and increase neural activity in neural diseases in which the pathogenic protein aggregates are intranuclear or diseases in which the protein aggregation is related to SBMA.

Neural diseases such as AD and ALS disease have the common characteristic of protein aggregates either inside neural cells in cytoplasm or in the extracellular space between two or more neural cells. This invention relates to a method for using a compound of formula I, II, III, IV, or GGA to inhibit the formation of the protein aggregates or alter the pathogenic protein aggregates into a non-pathogenic form. It is contemplated that this will attenuate some of the symptoms associated with these neural diseases.

In one embodiment the mammal is a human afflicted with a neural disease. In one embodiment of this invention, the negative effect of the neural disease being inhibited or reduced is ALS. ALS is characterized by a loss of functionality of motor neurons. This results in the inability to control muscle movements. ALS is a neurodegenerative disease that does not typically show intranuclear protein aggregates. It is contemplated that a compound of formula I, II, III, IV, or GGA will prevent or inhibit the formation of extracellular or intracellular protein aggregates that are cytoplasm, not intranuclear and not related to SBMA. It is also contemplated that a compound of formula I, II, III, IV, or GGA will alter the pathogenic protein aggregates into a form that is non-pathogenic. Methods for diagnosing ALS are commonly known to those skilled in the art. Additionally, there are numerous patents that describe methods for diagnosing ALS. These include U.S. Pat. No. 5,851,783 and U.S. Pat. No. 7,356,521 both of which are incorporated herein by reference in their entirety.

In one embodiment of the invention the negative effect of the neural disease being inhibited or reduced is AD. AD is a neurodegenerative disease that does not typically show intranuclear protein aggregates. It is contemplated that a compound of formula I, II, III, IV, or GGA will prevent or inhibit the formation of extracellular or intracellular protein aggregates. It is also contemplated that a compound of formula I, II, III, IV, or GGA will alter the pathogenic protein aggregates into a form that is non-pathogenic. Methods for diagnosing AD are commonly known to those skilled in the art. Additionally, there are numerous patents that describe methods for diagnosing AD. These include U.S. Pat. No. 6,130,048 and U.S. Pat. No. 6,391,553 both of which are incorporated herein by reference in their entirety.

In another embodiment, the mammal is a laboratory research mammal such as a mouse. In one embodiment of this invention, the neural disease is ALS. One such mouse model for ALS is a transgenic mouse with a Sodl mutant gene. It is contemplated that a compound of formula I, II, III, IV, or GGA will enhance the motor skills and body weights when administered to a mouse with a mutant Sodl gene. It is further contemplated that administering a compound of formula I, II, III, IV, or GGA to this mouse will increase the survival rate of Sodl mutant mice. Motor skills can be measured by standard techniques known to one skilled in the art. In yet another embodiment of this invention, the neural disease is AD. One example of a transgenic mouse model for AD is a mouse that overexpresses the APP (Amyloid beta Precursor Protein). It is contemplated that administering a compound of formula I, II, III, IV, or GGA to a transgenic AD mouse will improve the learning and memory skills of said mouse. It is further contemplated that a compound of formula I, II, III, IV, or GGA will decrease the amount and/or size of β-amyloid peptide and/or plaque found inside, between, or outside of neurons. The β-amyloid peptide or plaque can be visualized in histology sections by immunostaining or other staining techniques.

In one embodiment of the invention administering a compound of formula I, II, III, IV, or GGA to a mammal alters the pathogenic protein aggregate present into a non-pathogenic form. In another embodiment of the invention, administering a compound of formula I, II, III, IV, or GGA to a mammal will prevent pathogenic protein aggregates from forming

Another aspect of this invention relates to a method for reducing seizures in a mammal in need thereof, which method comprises administering the pharmaceutical compositions provided herein, thereby reducing seizures. The reduction of seizures refers to reducing the occurrence and/or severity of seizures. In one embodiment, the seizure is epileptic seizure. In another embodiment, the methods of this invention prevent neural death during epileptic seizures. The severity of the seizure can be measured by one skilled in the art.

In some embodiments, a pharmaceutical formulation described herein exerts cytoprotective effects on a variety of organs, e.g., the eye, brain and heart. (See, for example Ishii Y., et al., Invest Ophthalmol Vis Sci 2003; 44:198292; Tanito M, et al., J Neurosci 2005; 25:2396-404; Fujiki M, et al., J Neurotrauma 2006; 23:1164-78; Yasuda H, et al., Brain Res 2005; 1032:176-82; Ooie T, et al., Circulation 2001; 20; 104:1837-43; and Suzuki S, et al., Kidney Int 2005; 67:2210-20).

In certain aspects, the methods described herein relate to administering a compound of formula I, II, III, IV, or GGA or the isomeric compounds or compositions thereof in vitro. In other aspects the administration is in vivo. In yet other aspects, the in vivo administration is to a mammal. Mammals include but are not limited to humans and common laboratory research animals such as, for example, mice, rats, dogs, pigs, cats, and rabbits.

Compounds, compositions and methods of the invention described herein include the disclosures found in international application No.: WO 2012/031028 and the international PCT application entitled “GERANYLGERANYLACETONE DERIVATIVES”, filed on Feb. 29, 2012, both of which are incorporated herein in its entirety by reference. All citations herein are incorporated herein by reference in their entirety.

### EXAMPLES

### Example 1

### Transdermal Formulation

10 g of (5E, 9E, 13E) geranylgeranyl acetone and 50 g of oleic acid is dissolved in 100 ml propylene glycol by stirring. This solution is added to an aqueous acrylate adhesive dispersion while mixing. An adhesive thickener is added and the resulting mixture is stirred until homogenous. Then, the mixture is coated on an impermeable backing such as a polyester film laminate and dried. A release liner (e.g. a siliconized plastic sheet) is laminated to the adhesive layer. The final sheet is die cut to form transdermal devices of about 20 cm² comprising about 20 mg (5E, 9E, 13E) geranylgeranyl acetone, each of which is packaged individually.

### Example 2

### Development of Two Transdermal Patch Formulations Incorporating GGA to Two Developed Formulations—Thickness Control

The following data demonstrates that GGA can be administered over a prolonged period of time to achieve transdermal administration of GGA in accordance with this invention in patients suffering from neural disorders. Prior to adding GGA, a model was established to calculate wet film thickness and its relationship with dry film thickness, GGA amount, and the remaining excipients. This model allows one to input desired film thickness, from which the wet film weight and compositions are calculated for accurate sample preparation. Table 1 shows an example.

TABLE 1

| | |
|---|---|
| 4 cm × 7 cm Reference Dry Film Thickness (μm) | 160 |
| Reference Film Dry Volume (cm³) | 0.4480 |
| Reference Film Wet Volume (cm³) | 0.7434 |
| Desired Film Dry Thicknes (μm) | 160 |
| Desired Batch Size (gm) | 2.1 |
| Desired Film Wet Volume (cm³) | 0.7434 |
| Desired casting film Thickness (μm) | 265.5167 |
| Number of patches made (40% coverage) | 2.0176 |

| component | Dry film wt % | Wet film wt % |
|---|---|---|
| Duro Tak | 59.67 | 75.24 |
| Oleic acid | 13.99 | 8.38 |
| PVP | 19.64 | 11.77 |
| GGA | 6.70 | 4.01 |
| [mixture of | | |
| cis and trans] | | |
| SPAN80 | 1.00 | 0.60 |
| Total | 101.00 | 100.00 |

Duro Tak + Oleic Acid
Trial #4 + SPAN with GGA (2 4 × 7 patches)

| Component | Wet wt (gm) | Dry wt (gm) | Dry wt % |
|---|---|---|---|
| Duro Tak | 1.58 | 0.751 | 59.08 |
| Oleic acid | 0.176 | 0.176 | 13.86 |
| PVP | 0.247 | 0.247 | 19.45 |
| GGA | 0.084 | 0.084 | 6.63 |
| [mixture of | | | |
| cis and trans] | | | |
| SPAN80 | 0.013 | 0.013 | 0.99 |
| Total | 2.1 | 1.27 | 100 |
| OA/PV ratio | 0.712 | | |

### GGA Film Casting

Using the calculation scheme shown in Table 1, two GGA formulations (2 gram batches) were prepared. Both formulations were optically transparent. Trial #6 formulation was casted onto a 9744 release liner at 60° C. while #4 was casted at ambient temperature using tetrahydrofuran to adjust viscosity. Second batches of the similar formulations were prepared for tackiness, peeling, dissolution, and HPLC testing. In addition, two 4 cm×7 cm patches (one used #4 and the other #6 formulation) were prepared for Coyote's animal test.

### Tackiness and Adhesion Test of GGA Films (Two Formulations)

(a) a. Rolling Ball Tackiness Test

Following USP guideline of tackiness test using a rolling ball slider of 21.5 degree, we tested the tackiness of the trial #4 and #6 formulation and compared with a FDA approved adhesive backing (3 M 9699) to qualitatively evaluate the acceptance of the two prototype formulations. Table 2 shows the results.

TABLE 2
Rolling Ball Tackiness Test

| | Standard | | |
|---|---|---|---|
| Trials | (3M 9699 Backing) | Formulation #4 | Formulation #6 |
| 1 | 13 mm | 23 mm | 15 mm |
| 2 | 19 mm | 30 mm | 13 mm |
| 3 | 18 mm | 28 mm | 10 mm |
| 4 | 15 mm | 27 mm | 17 mm |
| 5 | 22 mm | 23 mm | 15 mm |
| 6 | 18 mm | 24 mm | 15 mm |
| 7 | 19 mm | 23 mm | 14 mm |
| 8 | 21 mm | 20 mm | 12 mm |
| 9 | 19 mm | 27 mm | 15 mm |
| 10 | 22 mm | 21 mm | 15 mm |
| Total | 186.0 mm | 246.0 mm | 141.0 mm |
| Average | 18.6 mm | 24.6 mm | 14.1 mm |
| Standard | 2.9 mm | 3.2 mm | 2.0 mm |
| Deviation | | | |

### 90 and 180 Degree Peeling Tests

Patches were adhered to stainless steel (#304) surface with 90 and 180 degree peeling angles with respect to the benchtop. By handing appropriate weight suitable for the standard (3M 9699), we qualitatively evaluate the peeling phenomena of the prototype formulations. Table 3 shows the results and our assessment.

TABLE 3
Peeling tests and their indications

| | | | |
|---|---|---|---|
| Backing | Standard (3M 9699 Backing) | Formulation #4 | Formulation #6 |

90 Degree Peeling Test

| | | | |
|---|---|---|---|
| Weight Used | Big Weight | Small Weight | Expected to be too adhesive to be |
| | | | peeled with the standard weights used |
| | | | here. |
| Result | After 2 minutes, only half was | After 1.5 min, | Expected to be too adhesive to be |
| | off. | only 40 mm was | peeled with the standard weights used |
| | | off. | here. |

| | |
|---|---|
| Remark | Since only half was peeled after 2 minutes, a 90 degree peeling test is not as |
| | representative as a 180 degree peeling test, which more represent human behavior when |
| | taking off a patch from the skin. |

180 Degree Peeling Test

| | | | |
|---|---|---|---|
| Weight Used | Small Weight | Small Weight | Big Weight |
| Result | 20.44 sec | 9.56 sec | After 3 minutes, only 4 mm was off. |

| Remark | #4 formulation is about half as adhesive as 3M 9699, which, on our skin test, | |
|---|---|---|
| | appears to be practical. #6 on the other hand could be a little too adhesive, but | |
| | still within the acceptable level according to the skin test. The other observations from | |
| | the peeling tests are that there is no residual formulation left on the skin nor the SS 304 | |
| | surface for the #4 formulation while #6 seems to leave small amount of formulation | |
| | after peeling. Nevertheless, these two formulations though not fully perfected can be | |
| | modified to become commercially acceptable formulations. | |
| | Small Weight | 54.22 g |
| | Big Weight | 104.18 g |

### HPLC Tests

### Dissolution

A 3.2 cm×3 cm patch was prepared (containing 10 mg GGA) and adhered to a 47 mm diameter PTFE Pall membrane. The assembly was then clipped to two pins to sink the membrane-patch assembly to the bottom of the vessel and remains at the fixed position. Sample was pulled and detected with HPLC refractive index detector set at the polarity mode for detecting GGA concentration and compared with a 0.05 mg/mL standard solution, which represents 50% release under our specific dissolution condition.

### Refractive Index Detector HPLC Test

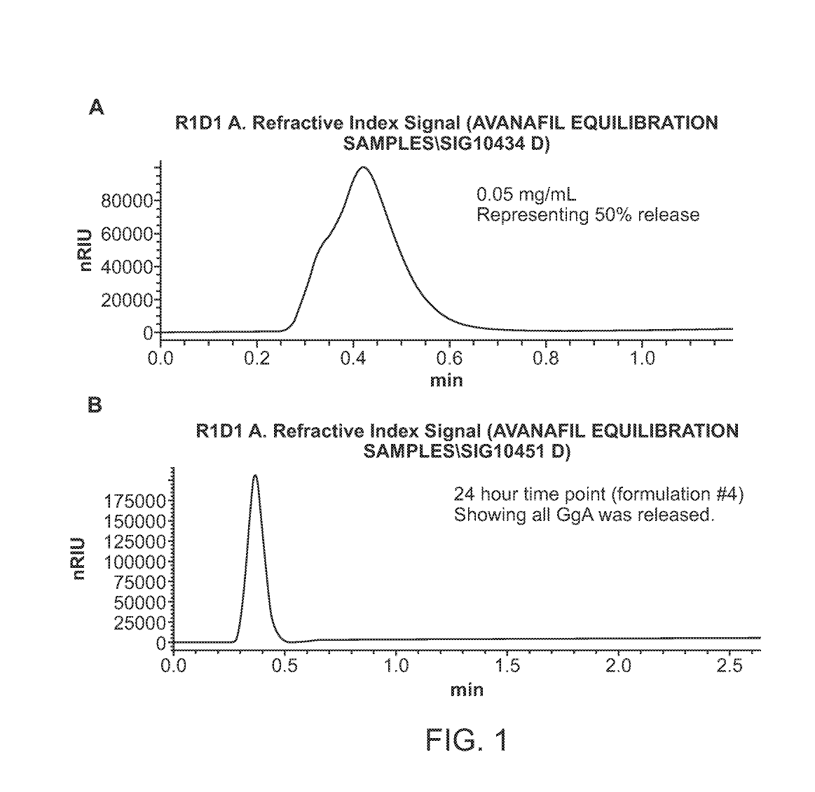

Two chromatograms (one standard of 0.05 mg/mL and one sample taken at 24 hour time point) in FIGS. 1A and 1B illustrate the dissolution results. Nearly all GGA in the membrane was released according to this specific dissolution setting (i.e., acetonitrile as the media).

The above data demonstrate that developing a GGA transdermal patch formulation is technically feasible with 3M's 9699 backing coupled with a 9744 release liner. Furthermore, oleic acid is a well known SPE and appeared to be miscible with a Duro Tak based matrix system containing the desired GGA amount.

### Example 3

### In Vitro Study of the Transdermal Permeation

An in vitro model using a vertical, two-compartment diffusion cell assembly mounted with freshly excised full thickness abdominal skin of hairless rats (Kfa credo) is used in this study. The donor solutions have a volume of 1.5 mL and comprised the compound of (5E, 9E, 13E) geranylgeranyl acetone, solvent and permeation enhancer. The receptor solution in all experiments consists of Hank's Balanced Salt Solution (Gibco BRL) with sodium azide. The skin permeation profile is followed for up to 50 h by sampling the receptor solution and assaying the drug concentration.

The ratio of the cumulated quantities detected in the receptor compartment to the skin area (ng/cm²) are plotted in function of the time and the flux of (5E, 9E, 13E) geranylgeranyl acetone through the skin in the presence of various solvents and skin permeation enhancers is deduced from the linear part of the plotted results.
Experiment 1: borate buffer pH 10, 0.1 M+(5E, 9E, 13E) geranylgeranyl acetone at 40 mg/mL
Experiment 2: borate buffer pH 10, 0.1 M (900 μl/l)+ethanol (95%; 100 μl/ml)+(5E, 9E, 13E) geranylgeranyl acetone (100 μg ml)
Experiment 3 : propylene glycol+eucalyptus oil (0%, 1%, 5%)+(5E, 9E, 13E) geranylgeranyl acetone 10 mg/ml
Experiment 4 : propylene glycol+oleic acid (OA) or lauric acid (LA) (0%, 5%)+(5E, 9E, 13E) geranylgeranyl acetone 10 mg/ml

### Example 4

### In Vitro Study of Transdermal Patch

Various in vitro methods or drug-release studies are used to determine the release of drug from transdermal patches, namely, the paddle over disc method (USP apparatus 5), the cylinder modified USP basket method (USP apparatus 6), and the reciprocating disc method (USP apparatus 7). The paddle over disc method (USP apparatus 5) is identical to the USP paddle dissolution apparatus, except that the transdermal system is attached to a disc or cell resting at the bottom of the vessel which contains medium at 32±5° C. The cylinder modified USP basket method (USP apparatus 6) is similar to the USP basket type dissolution apparatus, except that the system is attached to the surface of a hollow cylinder immersed in medium at 32±5° C. In reciprocating disc method (USP apparatus 7), patches attached to holders are oscillated in small volumes of medium, allowing the apparatus to be useful for systems delivering low concentration of drug.

In vitro permeation studies are conducted using specially designed diffusion cells such as Franz diffusion Cells, and Keshary-Chien cells. The transdermal patch is placed between the receptor compartment, and the donor compartment, and the amount of drug permeated at different time points is determined by a suitable analytical method.

Stability studies are conducted to investigate the influence of temperature and relative humidity on the drug content in different formulations. The transdermal formulations are subjected to stability studies as per ICH guidelines.

Skin hypersensitivity reaction or skin irritation of a transdermal patch is studied in white albino rat, mice or white rabbit models. A 0.8% v/v aqueous solution of formalin is used as standard irritant. The formalin treated groups are compared to drug and blank treated groups for edema and erythema.

### Example 5

### Pharmacokinetics of Transdermal Patch Formulation #4 with 30 mg GGA of Compound of Formula III in the Rat

The hair was removed on the back side of Sprague-Dawley rats to create a large bald area to allow complete naked skin contact of a 4 cm×7 cm transdermal patch formulation #4. Patches where attached and blood plasma samples were collected after 24 h, 72 h, 120 h, and 168 h. The blood plasma concentration of GGA was measured and is shown in table 4 below.

TABLE 4
Rat Pharmacokinetics with Transdermal Patch Formulation #4

| | Plasma Concentration of Compound of Formula III (ng/ml) | | | |
|---|---|---|---|---|
| | Rat 1 | Rat 2 | Rat 3 | Mean ± SD |
| Day 1 | 24.22 | 5.04 | 13.33 | 14.20 ± 9.62 |
| Day 3 | 10.24 | 0.00 | 0.00 | 3.41 ± 5.91 |
| Day 5 | 43.40 | 14.17 | 0.00 | 19.19 ± 22.13 |
| Day 7 | 9.07 | 0.00 | 0.00 | 3.02 ± 5.23 |

The Area under the Curve over 7 days (AUC_{0-168 hrs}) for transdermal patch formulation #4 was approximately 1668 ng*hr/ml.

### Example 6

### Pharmacokinetics of Transdermal Patch Formulation #6 with 30 mg GGA of Compound of Formula III in the Rat

The hair was removed on the back side of Sprague-Dawley rats to create a large bald area to allow complete naked skin contact of a 4 cm×7 cm transdermal patch formulation #6. Patches where attached and blood plasma samples were collected after 24 h, 72 h, 120 h, and 168 h. The blood plasma concentration of GGA was measured and is shown in table 5 below.

TABLE 5
Rat Pharmacokinetics with Transdermal Patch Formulation #6

| | Plasma Concentration of Compound of Formula III (ng/ml) | | | |
|---|---|---|---|---|
| | Rat 1 | Rat 2 | Rat 3 | Mean ± SD |
| Day 1 | 34.19 | 8.29 | 39.88 | 27.45 ± 16.84 |
| Day 3 | 0.00 | 20.67 | 10.51 | 10.39 ± 10.33 |
| Day 5 | 8.01 | 0.00 | 0.00 | 2.67 ± 4.62 |
| Day 7 | 12.53 | 11.53 | 15.86 | 13.31 ± 2.26 |

The Area under the Curve over 7 days (AUC_{0-168 hrs}) for transdermal patch formulation #6 was approximately 1935 ng*hr/ml.

### Example 7

### In Vivo Study of Transdermal Patch

In vivo studies are performed in animal or human models. The most common animal species used are mouse, hairless rat, hairless dog, hairless rhesus monkey, rabbit, guinea pig.

Plasma concentrations and PK parameters are obtained for transdermal formulation PK studies. PK PARAMETERS are calculated from noncompartmental analysis (NCA) model using WinNonlin software and the linear/log trapezoidal method. If necessary, the concentration-time points are manually selected for use in the calculation.

$$Bioavailability$$

$$F ( \% ) = Bioavailability - \frac{AUC ( PO ) / Dose ( PO )}{AUC ( PO ) / Dose ( PO )} \times 100$$

The compositions of this invention are tested in vivo for their ability to alleviate neurodegenerations induced by Kainic acid. See, for example, international application No. PCT/US2011/050071, supra. A composition of this invention is dosed to Sprague-Dawley rats, and Kainic acid is injected. Seizure behaviors are observed and scored (see, e.g., R. J. Racine, Modification of seizure activity by electrical stimulation: II. Motor seizure, Electroencephalogr. Clin. Neurophysiol. 32 (1972) 281-294). Brain tissues of rats are sectioned on histology slides, and neurons in hippocampus tissues are stained by Nissl.

Phase I clinical trials are conducted in human subjects to determine the safety, Phase II studies are conducted to determine short term safety, and mainly effectiveness in patients. Phase III trials are conducted in large number of patient population to study the safety and effectiveness. Phase IV trials or post marketing surveillance studies are conducted to detect adverse drug reactions of marketed patches.

1. A method for treatment of a neural disease, disorder or condition, the method comprising administering to a subject suffering from said neural disease, disorder, or condition, a therapeutically effective amount of (5E, 9E, 13E) geranylgeranyl acetone, wherein (5E, 9E, 13E) geranylgeranyl acetone is administered to said subject by transdermal application and the (5E, 9E, 13E) geranylgeranyl acetone is present in a ratio of greater than 90:10 of (5E, 9E, 13E) to (5Z, 9E, 13E) geranylgeranyl acetone isomers.

2. A method for inducing expression of a heat shock protein in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application and the (5E, 9E, 13E) geranylgeranyl acetone is present in a ratio of greater than 90:10 of (5E, 9E, 13E) to (5Z, 9E, 13E) geranylgeranyl acetone isomers.

3. A method for inhibiting neural death in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application and the (5E, 9E, 13E) geranylgeranyl acetone is present in a ratio of greater than 90:10 of (5E, 9E, 13E) to (5Z, 9E, 13E) geranylgeranyl acetone isomers.

4. The method of claim 1, wherein the (5E, 9E, 13E) geranylgeranyl acetone is essentially free of or is free of (5Z, 9E, 13E) geranylgeranyl acetone.

5. A medical patch for the rate-controlled transdermal administration of (5E, 9E, 13E) geranylgeranyl acetone through intact skin for an extended period of time which comprises:
(a) a drug reservoir comprising (5E, 9E, 13E) geranylgeranyl acetone and a permeation enhancer in amounts sufficient to deliver (5E, 9E, 13E) geranylgeranyl acetone at a therapeutically effective rate for said extended period of time; and
(b) a drug-impermeable backing laminate.

6. The medical patch of claim 5, wherein (5E, 9E, 13E) geranylgeranyl acetone is present in a ratio of greater than 80:20 of (5E, 9E, 13E) to (5Z, 9E, 13E) geranylgeranyl acetone isomers.

7. The medical patch of claim 5, wherein (5E, 9E, 13E) geranylgeranyl acetone is present in a ratio of greater than 90:10 of (5E, 9E, 13E) to (5Z, 9E, 13E) geranylgeranyl acetone isomers.

8. The medical patch of claim 5, wherein the permeation enhancer is ethanol, glycerol monocaprylate, glycerol monolaurate, glyceryl monooleate, hydroxypropyl β-cyclodextrin, lauric acid, myristic acid, oleic acid, oleyl alcohol, palmitic acid, polyethylene glycol, propylene glycol, sodium lauryl sulfate(SLS), steric acid, tween 80 or combination thereof.

9. An apparatus for treatment of neural disease, disorder or condition, comprising an adhesive patch for being adhered to the skin of a subject suffering from the neural disease, disorder or condition, a therapeutically effective amount of (5E, 9E, 13E) geranylgeranyl acetone wherein the compound is dispersed on said patch; and at least one permeation enhancer for introducing said compound from the patch into the body of the subject.

10. The apparatus of claim 9, wherein (5E, 9E, 13E) geranylgeranyl acetone is present in a ratio of greater than 80:20 of (5E, 9E, 13E) to (5Z, 9E, 13E) geranylgeranyl acetone isomers.

11. The apparatus of claim 5, wherein (5E, 9E, 13E) geranylgeranyl acetone is present in a ratio of greater than 90:10 of (5E, 9E, 13E) to (5Z, 9E, 13E) geranylgeranyl acetone isomers.

12. The apparatus of claim 9, wherein the permeation enhancer is ethanol, glycerol monocaprylate, glycerol monolaurate, glyceryl monooleate, hydroxypropyl β-cyclodextrin, lauric acid, myristic acid, oleic acid, oleyl alcohol, palmitic acid, polyethylene glycol, propylene glycol, sodium lauryl sulfate(SLS), steric acid, tween 80 or combination thereof

13. The apparatus of claim 9, wherein the neural disease, disorder or condition is Alzheimer's disease, amyotrophic lateral sclerosis disease, Parkinson's disease, or multiple sclerosis.

14. A method for treatment of a neural disease, disorder or condition, the method comprising administering to a subject suffering from said neural disease, disorder, or condition, a therapeutically effective amount of (5E, 9E, 13E) geranylgeranyl acetone, wherein (5E, 9E, 13E) geranylgeranyl acetone is administered to said subject by transdermal application comprising a medical patch of claim 5.

15. A method for inducing expression of a heat shock protein in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application comprising a medical patch of claim 5.

16. A method for inhibiting neural death in a subject in need thereof, the method comprising administering to the subject a therapeutically effective amount of (5E, 9E, 13E) geranylgeranyl acetone, wherein (5E, 9E, 13E) geranylgeranyl acetone is administered to said subject by transdermal application comprising a medical patch of claim 5.
